(19) United States
(12) Patent Application Publication
(10) Pub. No.: US 2010/0312485 A1
(43) Pub. Date: Dec. 9, 2010

(54) Method and arrangement for determination of the individual incretin sensitivity index of a subject

(76) Inventors: Eckhard Salzsieder, Karlsburg (DE); Petra Augstein, Freest (DE)

Correspondence Address: COLLARD & ROE, P.C., 1077 NORTHERN BOULEVARD, ROSLYN, NY, 11576, US

(21) Appl. No.: 12/802,434

(22) Filed: Jun. 7, 2010

(30) Foreign Application Priority Data
Jun. 8, 2009 (DE) 10 2009 024 229.5

(51) Int. Cl. G06F 19/00; G01N 33/48

(52) U.S. Cl. 702/19

(57) ABSTRACT

A computer-aided method for determination of a subject's individual incretin sensitivity index determines the individual metabolic situation in the form of the personal metabolic situation of the subject. The individual incretin effect factor and the effect found of the administered incretin mimetic or incretin enhancer by simulation for the mean deviation of the daily glycemia are determined with the personal metabolic situation data and recorded numerically. The corresponding effect factor of a slow-acting insulin, whose step-by-step administration is varied until the mean deviation of the daily glycemia is identical to the mean deviation of the daily glycemia determined previously by the incretin mimetic or incretin enhancer, is determined. The insulin dosage is recorded numerically. The individual incretin sensitivity index is calculated from the relationship between the dosages of the incretin mimetic or incretin enhancer determined for identical deviation of the daily glycemia and the respectively individually different insulin dosage.

### CROSS REFERENCE TO RELATED APPLICATIONS

Applicants claim priority under 35 U.S.C. §119 of German Application No. 10 2009 024 229.5 filed Jun. 8, 2009.

### BACKGROUND OF THE INVENTION

1. Field of the Invention

The invention relates to a method and an arrangement for computer-aided automatic determination of the individual incretin sensitivity index of a subject. The invention is used in computer-aided expert systems for health management, health services and in health care.

2. Description of the Related Art

Diabetes mellitus is a group of metabolic illnesses which are characterized by increased blood glucose levels (hyperglycemia). Hyperglycemia is the result of an absolute or relative shortage of insulin, caused by a reduced number of beta cells, an insulin secretion disturbance and/or reduced insulin effect. The majority of diabetes cases can be subdivided into two categories, Type 1 diabetes and Type 2 diabetes, with about 90-95% of cases being Type 2 diabetes.

Jay S. Skyler; Diabetes Mellitus: Pathogenesis and Treatment Strategies, in the Journal of Medical Chemistry, 2004, Volume 47, 4113-4117 has described that Type 2 diabetes is normally caused by increasing insensitivity to insulin (insulin resistance) and by cessation of the secretary response to glucose.

Insulin has a key function in the control of carbohydrate and lipid metabolism. When glucose is administered after consuming fluids containing carbohydrates, and is absorbed into the blood, the increased blood glucose concentration stimulates the release of insulin. Insulin allows the glucose to enter muscle tissue and various other tissues by activation of glucose transporters. Insulin also stimulates the liver, in order to store glucose in the form of glycogen. When the blood glucose concentration falls, the glucose-stimulated insulin secretion ceases.

Insulin also has important effects on lipid metabolism. In a healthy individual, lipolysis is constrained. In a Type 2 diabetic, the increased amount of free fatty acids leads to stimulation of lipolysis and glyconeogenesis.

Insulin therefore plays a critical role in the control of carbohydrate and lipid metabolism. Absolute and/or relative lack of insulin secretion results in disastrous effects on organs and tissues. Diabetes mellitus, which is the commonest and most important metabolic human illness, is fundamentally a disturbance in insulin secretion and insulin effect.

Type 1 diabetes or insulin-dependent diabetes mellitus is the result of an immune-mediated destruction of the pancreatic cells, of the beta cells, with the consequence of a complete lack of insulin, and the resultant need to substitute insulin.

Type 2 diabetes, or non-insulin-dependent diabetes mellitus, is a complex syndrome of insulin resistance and insulin secretion. Over time, it can lead to long-term damage, to functional disturbances or to failure of various organs, particularly the eyes, the kidneys and the cardiovascular system.

The already known pharmaceutic products, which are used to treat Type 2 diabetes, include, inter alia, insulin, biguanides, sulfonylureas and thiazolidindiones. Because of the natural progress in insulin resistance and beta cell functional disturbance over the course of the Type 2 diabetes illness, most diabetes patients require insulin therapy once their illness has lasted for a greater or lesser time. The main disadvantages of antidiabetics which can be administered orally (OAD) is that the glycemia profiles in some cases fluctuate severely, an increase in weight and the formation of edemas. In addition, none of these means offers the potential to maintain the function of the insulin-producing beta cells in the pancreas in the long term.

Incretin hormones are hormones which result in an increase in the amount of secreted insulin in relation to food-dependent glucose deviation. These incretin hormones furthermore have effects on glucose secretion, stomach emptying and the resorption rate of the food consumed. They can also improve insulin sensitivity. They also have a protective effect on the insulin-producing beta cells, by inhibiting necrosis.

It is known that the blood glucose level is also regulated to a major extent by the incretins. A therapy principle has therefore been developed in diabetology which provides a reactivation of the incretin effect, which decreases in a subject who is ill with Type 2 diabetes.

Incretin-hormone glucose-dependent insulinotropic polypeptide (GIP) and glucagon-like peptide 1 (GLP-1) are known.

These intestinal hormones are released into the blood circulation after oral consumption of carbohydrates and result in various processes that reduce the blood glucose.

The incretins dock on the islet cells, the alpha and beta cells, of the pancreas.

Goke, et al., in J. Biol. Chem., 268, 19650-55, 1993 describes that, there, the incretins result in various effects, depending on the instantaneous blood glucose. The incretins stimulate the beta cells in the pancreas to release insulin. Furthermore, they stimulate the growth of the beta cells, which produce the insulin in the pancreas.

According to Orskov et al., in Diabetes 42, 658-61, 1993, and D'Alessio, et al., in J. Clin. Invest., 97, 133-138, 1996, incretins in the alpha cells of the pancreas inhibit the formation of glucagon which is known to be an antagonist of insulin.

Willms, B. et al., in J. Clin. Endocrinol Metab 81 (1), 327-32, 1996, has disclosed that glucose production in the liver (hepatic gluconeogenesis) is restricted by the incretins. This glucose production is responsible for the increased fasting glucose.

Furthermore, the incretins delay stomach emptying, as a result of which foodstuffs enter the blood more slowly, making it easier to control the blood glucose. They also enhance the sense of fullness in the subject, which can lead to a reduction in body weight.

Incretins are responsible for 60 to 70% of the total insulin secretion after consumption of carbohydrates. They ensure that the pancreas releases much more insulin after absorption of glucose from the intestine than after infusion of the same amount of glucose directly into the bloodstream. Incretin results in a decrease in the blood glucose concentration.

However, the effect of the body's own incretin GLP-1 is limited by being degraded by the proteolytic enzyme dipeptidyl-peptidase-4 (DPP-4). The enzyme DPP-4 converts the protein hormone GLP-1 to an ineffective molecule in a few minutes in the organism of the subject.

Nauck, M. A. in Diabetologie/Inkretinmimetika and Inkretinverstärker, 2007, Vol 3, No. 5, 387-398, Springer Science+Business Media, describes that the active pharmaceutical ingredients called incretin mimetics can be used, by virtue of their incretin effect, to treat diabetics.

Synthetically produced incretin mimetics are structural analogs of the incretins GLP-1 and, like them, bind to the GLP-1 receptor. Their effects therefore correspond to those of the incretins. However, they are resistant to the DPP-4 enzyme.

The incretin mimetics have the capability to mimic the effect of the body's own hormone GLP-1, whose blood-glucose-reducing characteristics are referred to, for short, as the incretin effect. When administered, the GLP-1 is not enzymatically broken down by DPP-4, and therefore acts for a longer time.

Synthetic incretin mimetics are exenatide, which is derived from the toxin of the saliva of a type of American lizard, or liraglutide, which is derived from GLP-1. When administered, the analog is enzymatically broken down more slowly by DPP-4, and therefore acts for a longer time.

WO 2000/041546 A2 and EP 1140145 B1 have disclosed the pharmaceutical formulation of the synthetically produced polypeptide exendin and of the exendin agonist peptide and their production and use for increasing the sensitivity of an individual to exogenous or endogenous insulin. The incretin mimetic described is the pharmaceutical agent exendin-4. When this incretin mimetic is used, insulin is released at high blood glucose levels. The aim of this is to achieve quasi-physiological blood glucose control, and this explains why the risk of hypoglycemia is low when incretins are externally supplied.

WO 1998/30231 A1 has disclosed the formulations of the incretin mimetic exendin and its agonist, and their therapeutic use for treatment of Type 2 diabetic illnesses, and to reduce the consumption of foodstuffs, as well as to reduce obesity.

WO 2009/024015 A1 has disclosed an incretin mimetic in the form of a consistent recipe for synthetic exenatide, the production method and the use of the exenatide, and a method for determining the content of exenatide in the recipe. The pharmaceutical agent has an increasingly significant and long stability. Exenatide is likewise used to treat Type 2 diabetes, and satisfies the conditions that the blood glucose is reduced, stomach emptying is delayed, and the absorption of carbohydrates into the blood is slowed down.

For the individual treatment of subjects with Type 2 diabetes, it is a daily or even hourly requirement to keep the blood glucose level in the normal range.

Continuous glucose monitoring (CGM) offers a basis for new therapeutic options for diabetes treatment.

People with diabetes mellitus can therefore now see their current glucose level, and the development of the glucose level. Alarms in the event of rapid changes in the glucose concentration and on departure from the individual glucose target range allow an early reaction, before acute problems arise.

A wide range of systems for continuous glucose monitoring (CGM) are known, which continuously measure the glucose level around the clock. The systems store a glucose value every 5 minutes, and emit an alarm in the event of excess glucose (hyperglycemia) or inadequate glucose (hypoglycemia). Continuous long-term blood glucose measurement is carried out over 3 to 5 days. A flexible sensor needle is connected to a small transmitter which sends the sensor data to a monitor, stores it there, and produces it for subsequent evaluation. This therefore also allows real-time recording of the hyperglycemic and hypoglycemic glucose fluctuations.

The model-based program KADIS has been disclosed by Rutscher et al.: KADIS—a Computer-Aided Decision Support System for Improving the Management of Type 1-Diabetes, in Exp. Clin. Endocrinol., Vol. 95, No. 1, 1990, pp. 137-147, and Salzsieder, E., et al: Computer-aided systems in the management of Type 1-diabetes: the application of a model-based strategy, in Computer Methods and Programs in Biomedicine, 32 (1990), pp. 215-224, Elsevier.

The interaction between the invasive continuous glucose monitoring system (CGMS) and the KADIS-based simulation program for optimization of blood glucose control is disclosed in Salzsieder, E.; Augstein, P., et al.: Telemedicine-Based KADIS Combined with CGMS has high Potential for Improving Outpatient Diabetes Care, in Journal of Diabetes Science and Technology, Vol. 1, pp. 511-521.

EP 1836667 A3 has disclosed a method and arrangement for computer-aided determination of the characteristic daily profile (CTP) of the individual glucose metabolism. The computer-aided determination of the personal CTP of each individual consists in producing a typical image of the current glucose metabolism situation, as a personal metabolic fingerprint of the individual glucose metabolism, with causally justified relationships between the individual daily profile of the blood glucose concentration and the endogenous and exogenous manipulated and controlled variables which influence these, in a qualitative and quantitative form, for the respective individual, by means of a model-based identification program. If the personal CTP of the blood glucose concentration of an individual is determined as a personal metabolic fingerprint, the determined CTP of the blood glucose concentration can be used as the basis for analyzing causally justified reasons for the profile of the blood glucose concentration, and for analyzing individual-specific information related to the specific influencing of the daily profile of the blood glucose concentration for this individual, with computer assistance.

For this purpose, continuous 72-hour glucose monitoring is first of all carried out for Type 1 or Type 2 diabetics, and a daily blood glucose profile determined from this for each diabetic is written to the model-based program. This computer-aided program results in a personal CTP being created for each diabetic, which for the first time links the blood glucose profile to the therapeutic measures and to the individual metabolism behavior of the diabetic. Similar to a DNA analysis, which produces the individual genetic fingerprint, the CTP is therefore the metabolic fingerprint of each diabetic. One precondition for the creation of the CTP is participation in the continuous 72-hour glucose monitoring, by means of sensors, and documentation of therapy and self-monitoring data, during the monitoring process. Every diabetic can use the personal CTP to more quickly and reliably identify and analyze his weaknesses in metabolism adjustment. In addition to the daily insulin requirement, tablet therapy, bread units consumed and sporting activities, the KADIS-based in-silico simulation system calculates the individual effect curves for insulin and sport as well as the 24-hour nutrition profile, and relates them to the measured daily blood glucose profiles. The model-based KADIS program allows the subject's own insulin profile to be calculated for Type 2 diabetes, to be displayed and to be included in the assessment of the CTP.

### SUMMARY OF THE INVENTION

The object of the invention is to provide a method for individually matched estimation of the potential therapeutic effectiveness of the active pharmaceutical ingredient incretin mimetic or incretin enhancer for therapeutic treatment of Type 2 diabetics.

The object of the invention is achieved by a method and an automation arrangement for computer-aided determination of the individual incretin sensitivity index of a subject by means of an automated in-silico simulation strategy with the result that protected knowledge can be obtained with regard to the response capability of the subject to incretin mimetic or incretin enhancer, and their effect, on the basis of therapeutic administration.

The method according to the invention starts with the determination of the individual metabolic situation in the form of the personal metabolic situation of the subject from patient base data, self-monitoring data and results from the CGM by means of a model-based indication program, followed by determination of the individual incretin effect factor from the data from the personal metabolic situation and with the effect determined of the administered incretin mimetic or incretin enhancer, by simulation, for the mean deviation in the daily glycemia, which is recorded numerically, and then the corresponding effect factor of a slow insulin, whose step-by-step administration is varied until the mean deviation of the daily glycemia is identical to the mean deviation in the daily glycemia determined previously by the incretin mimetic or incretin enhancer, is determined, and the insulin dosage is recorded numerically and, finally, the incretin sensitivity index is calculated from the relationship between the dosages of the incretin mimetic or incretin enhancer determined for identical deviation of the daily glycemia and the respectively individually different insulin dosage.

If the value of the incretin sensitivity index is greater than 1, the subject is considered to be incretin-sensitive.

The arrangement according to the invention for carrying out the method by means of automated in-silico simulation strategy includes on the input side, a first series circuit, in which the inputs of a data input module for the data signal for the continuously measured daily glucose profile (CGM), the data signal for the subject base data (PBD) and the data signal for the self-monitoring data (SKD), a downstream anonymization and memory module for allocation of the subject identity and for storage thereof in the patient data memory, an identification module which produces the characteristic daily profile (CTP), to whose second input a model module in which the mathematical model for describing the physiological glucose metabolism and the iteration program are stored, is coupled, and an incretin effect calculation module, to whose second input an incretin data input and memory module which produces the data signal for the dosage of the incretin mimetic or incretin enhancer, is connected, are connected in series. The output of the incretin effect calculation module, which generates the data signals for the daily glucose profile of the incretin effect (GTP_{GLP}) and the mean deviation of the blood glucose concentration for the incretin effect (MBG_{GLP}) are generated, is coupled to the first input of a comparator module. An insulin data input and memory module, which produces the data signal for the insulin dosage, is connected upstream of the first input of an insulin effect calculation module. From the output of the insulin effect calculation module, the data signals generated by it for the daily glucose profile for the insulin effect (GTP_{INS}) and the mean deviation of the blood glucose concentration (MBG_{INS}) are applied to the second input of the comparator module. The first output of the comparator module for the no decision is fed back for dosage equality of incretin and insulin via the insulin dose change module to the second input of the insulin effect calculation module. The input of an incretin effect equivalent calculation module is connected to the second output of the comparator module for the yes decision for dosage equality of incretin and insulin, and its output produces the data signal for the daily glucose profile of the insulin effect profile (GTP_{GLPequ}), which is equivalent to the incretin effect, for the downstream calculation module for the incretin effect equivalent insulin dose (INS_{equ}). The output of the calculation module for the incretin effect equivalent insulin dose (INS_{equ}), which produces the data signal for the incretin effect equivalent insulin dose (INS_{equ}), is connected to the input of the incretin sensitivity index calculation module in that the output of the incretin sensitivity index calculation module, which produces the data signal for the incretin sensitivity index (ISI), is connected to the input of the downstream output module for the incretin sensitivity index. The output module for the incretin sensitivity index, after its individual assessment of the incretin sensitivity index, as carried out by itself, produces at its output the data signal for the assessed incretin sensitivity index (ISI_{B}) as a process result of the automated in-silico simulation strategy.

### BRIEF DESCRIPTION OF THE DRAWINGS

Other objects and features of the present invention will become apparent from the following detailed description considered in conjunction with the accompanying drawings. It is to be understood, however, that the drawings are designed as an illustration only and not as a definition of the limits of the invention.

In the drawings,

The sole FIGURE shows a microcomputer arrangement for carrying out an automated in-silico simulation strategy in accordance with an embodiment of the invention.

### DETAILED DESCRIPTION OF PREFERRED EMBODIMENTS

The invention is described in more detail in the following text with reference to one exemplary embodiment.

The incretin sensitivity index is intended to be used as an individual-specific measure for the glucose-reducing effect to be expected from an incretin mimetic, to be administered therapeutically, for one subject.

The incretin effect factor is described in that, on average in the case of a subject with Type 2 diabetes, the therapeutic administration of 1 μg of incretin mimetic corresponds to the glucose-reducing effect of 0.68 IE of a slow-acting insulin.

The method according to the invention comprises an algorithm in which the individual metabolic situation is first of all determined for a subject with Type 2 diabetes, and whose incretin sensitivity index is intended to be determined. Continuous glucose long-term monitoring is carried out over at least 72 hours in everyday conditions, and the subject's personal metabolic situation is determined from the data by means of a model-based identification program. This personal metabolic situation is then displayed automatically on a personal computer, in the form of an in-silico image. The personal metabolic situation represents the characteristic 24-hour glucose profile of the subject with Type 2 diabetes, related to his individual endogenous and exogenous influencing factors.

The individual incretin effect factor is determined in-silico on the basis of the personal metabolic situation by simulation by testing the effect of 20 μg of incretin mimetic, which is administered in two doses of 10 μg each at 0600 hrs and at 1800 hrs, with computer assistance, and by numerically determining the mean deviation of the daily glycemia in the case of the incretin effect (MBG_{GLP}).

Furthermore, that insulin dose which corresponds to the previously determined deviation in the daily glycemia when 20 μg of the incretin mimetic is administered is determined by simulation. In this case, instead of testing the incretin mimetic, the effect of a slow-acting insulin is tested, which is likewise administered in two doses at 0600 hrs and at 1800 hrs, with the insulin doses being titrated up in steps of 0.5 IE until a deviation has been achieved which is identical to the mean deviation of the daily glycemia MBG_{INS} determined previously by the incretin mimetic, and the daily glycemia profiles GTP_{GLP} and GTP_{INS} have been made to match as well as possible. The corresponding insulin dosage INS_{equ} is recorded numerically.

Finally, the individual incretin sensitivity index ISI is determined by relating the dosages, determined by identical deviation in the daily glycemia, of the incretin mimetic of 20 μg and the respectively individually different insulin dosage INS_{equ}.

If the nondimensional value of the individual incretin sensitivity index ISI is greater than unity, it can be assumed that the subject with Type 2 diabetes is incretin sensitive. If the index value is less than unity, then this subject with Type 2 diabetes can be expected to react less sensitively to the therapeutic administration of an incretin mimetic. The assessed incretin sensitivity index ISI_{B} is the result of the automated, computer-aided determination of the individual incretin sensitivity index.

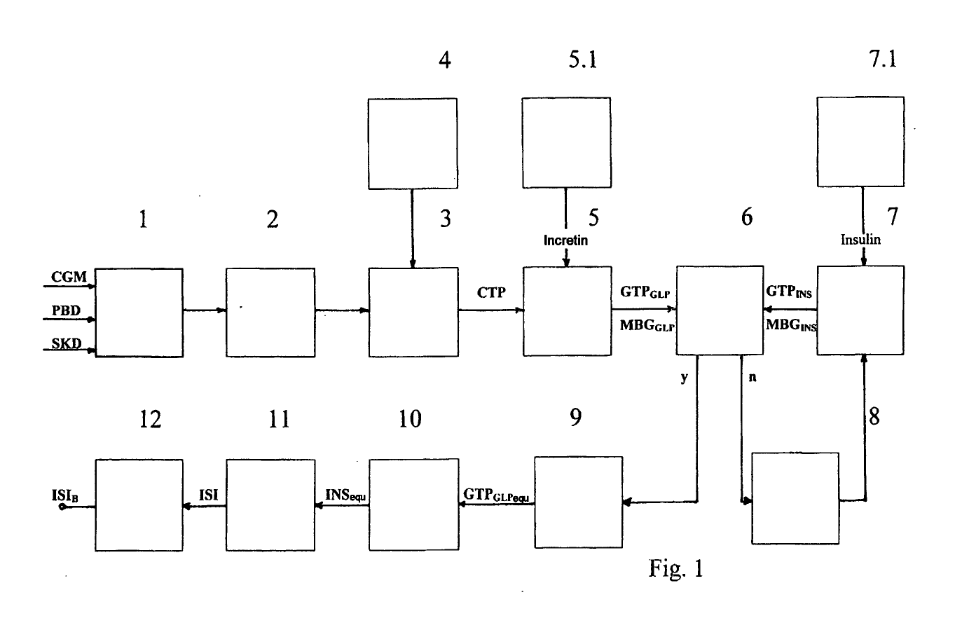

The method according to the invention is carried out by means of an automated in-silico simulation strategy, using a microcomputer arrangement. This arrangement will be described in the following text, using the drawing as shown in FIG. 1 for more detailed explanation.

The data signals in this arrangement mean:
CGM Continuously measured daily glucose profiles
PBD Subject base data: age, body size, body weight, type of diabetes
SKD Self-monitoring data: medication, nutrition, physical activities
CTP Personal daily characteristic profile as an in-silico image of the current metabolic situation of the relevant subject
GTP_{GLP} Daily glucose profile of the incretin effect
MBG_{GLP} Mean deviation in the blood glucose concentration with the incretin effect
GTP_{INS} Daily glucose profile of the insulin effect
MBG_{INS} Mean deviation in the blood glucose concentration with the insulin effect
GTP_{GLPequ} Daily glucose profile of the insulin effect profile equivalent to the incretin effect
INS_{equ} Insulin dose equivalent to the incretin effect
ISI Incretin sensitivity index
ISI_{B} Individually assessed incretin sensitivity index

The arrangement according to the invention for carrying out the automated in-silico simulation strategy consists, on the input side, of a first series circuit of the data input module 1, the anonymization and memory module 2, the identification module 3 with the model module 4 connected to it, the incretin effect calculation module 5 with the incretin data input and memory module 5.1 connected to it.

The data signal CMG is applied to the first input of the data input module 1; the data signal PBD is applied to the second input of the data input module 1, and the data signal SKD is applied to the third input of the data input module 1.

The anonymization and memory module 2 is used to allocate the subject identity, and to store this in the internal patient memory.

The model module 4 in which the mathematical model for describing the physiological glucose metabolism and the iteration program for controlling the step-by-step matching of the dosage equality of insulin and incretin mimetic is stored, is coupled to the second input of the identification module 3 which, at its output, produces the determined CTP as a data signal for passing onto the first input of the incretin effect calculation module 5. The second input of the incretin effect calculation module 5 is connected to the output of the incretin data input and memory module 5.1, in order to produce the data signal for incretin mimetic dosage.

The output of the incretin effect calculation module 5 is connected to the first input of the comparator module 6 in order to produce the data signals GTP_{GLP} and MBG_{GLP}.

The output of the insulin effect calculation module 7 is connected to the second input of the comparator module 6 in order to produce the data signals GTP_{INS} and MBG_{INS}. The first input of the insulin effect calculation module 7 is connected to the insulin data input and memory module 7.1 in order to produce the data signal for insulin dosage.

The first output of the comparator module 6 produces the decision n (no) for dosage equality of insulin and incretin mimetic, and is coupled via the insulin dose changing module 8 to the second input of the insulin effect calculation module 7.

The input of the second series circuit, whose input forms the incretin effect equivalent calculation module 9, is connected to the second output of the comparator module 6, which produces the decision y (yes) for dosage equality of insulin and incretin mimetic.

The calculation module for the incretin effect equivalent insulin dose 10, the incretin sensitive index calculation module 11 and the output module for the incretin sensitivity index 12 is then connected to the incretin effect equivalent calculation module 9.

At its output, the incretin effect equivalent calculation module 9 produces the data signal GTP_{GLPequ} for the downstream calculation module for the incretin effect of the equivalent insulin dose INS_{equ} 10. The output of the calculation module for the incretin effect of the equivalent insulin dose 10 produces the data signal INS_{equ} analogously to the incretin effect of the equivalent insulin dose, and is connected to the input of the incretin sensitivity index calculation module 11. The output of the incretin sensitivity index calculation module 11, which produces the data signal ISI, is connected to the input of the downstream output module for the incretin sensitivity index 12 and, after individual assessment of the incretin sensitivity index, the output at the end of the second series circuit produces the output data signal as the individually assessed incretin sensitivity index ISI_{B}, as the process result of the automated in-silico simulation strategy.

1. Method for determination of the individual incretin sensitivity index of a subject, preceded by computer-aided determination of the individual metabolic situation in the form of the personal metabolic situation of the subject, wherein with the data of the personal metabolic situation, the individual incretin effect factor and the established effect of the administered incretin mimetic are determined and recorded numerically by simulation for the mean deviation in the daily glycemia, and then the corresponding effect factor of a slow-acting insulin, whose step-by-step administration takes place until the mean deviation of the daily glycemia is identical to the mean deviation of the daily glycemia determined previously by the incretin mimetic or incretin enhancer, is determined, and the insulin dosage is recorded numerically, and the incretin sensitivity index is calculated from the relationship between the dosages of the incretin mimetic or incretin enhancer determined for identical deviation of the daily glycemia and the respectively individually different insulin dosage.

2. Method for determination of the individual incretin sensitivity index of a subject according to claim 1, wherein the method is carried out by means of automated in-silico simulation strategy.

3. Method for determination of the individual incretin sensitivity index of a subject according to claim 1, wherein the subject is considered to be sensitive to incretin if the value of the incretin sensitivity index is greater than 1.

4. Arrangement for determination of the individual incretin sensitivity index of a subject, wherein, on the input side, the arrangement comprises a first series circuit, in which the inputs of a data input module (1) for the data signal for the continuously measured daily glucose profile (CGM), the data signal for the subject base data (PBD) and the data signal for the self-monitoring data (SKD), a downstream anonymization and memory module (2) for allocation of the subject identity and for storage thereof in the patient data memory, an identification module (3) which produces the characteristic daily profile (CTP), to whose second input a model module (4) in which the mathematical model for describing the physiological glucose metabolism and the iteration program are stored, is coupled, and an incretin effect calculation module (5), to whose second input an incretin data input and memory module (5.1) which produces the data signal for the dosage of the incretin mimetic or incretin enhancer, is connected, are connected in series, wherein the output of the incretin effect calculation module, which generates the data signals for the daily glucose profile of the incretin effect (GTP_{GLP}) and the mean deviation of the blood glucose concentration for the incretin effect (MBG_{GLP}) are generated, is coupled to the first input of a comparator module (6), wherein an insulin data input and memory module (7.1), which produces the data signal for the insulin dosage, is connected upstream of the first input of an insulin effect calculation module (7), wherein, from the output of the insulin effect calculation module, the data signals generated by it for the daily glucose profile for the insulin effect (GTP_{INS}) and the mean deviation of the blood glucose concentration (MBG_{INS}) are applied to the second input of the comparator module, wherein the first output of the comparator module for the no decision is fed back for dosage equality of incretin and insulin via the insulin dose change module (8) to the second input of the insulin effect calculation module, wherein the input of an incretin effect equivalent calculation module (9) is connected to the second output of the comparator module for the yes decision for dosage equality of incretin and insulin, and its output produces the data signal for the daily glucose profile of the insulin effect profile (GTP_{GLPequ}), which is equivalent to the incretin effect, for the downstream calculation module for the incretin effect equivalent insulin dose (INS_{equ}) (10), wherein the output of the calculation module for the incretin effect equivalent insulin dose (INS_{equ}), which produces the data signal for the incretin effect equivalent insulin dose (INS_{equ}), is connected to the input of the incretin sensitivity index calculation module (11) in that the output of the incretin sensitivity index calculation module, which produces the data signal for the incretin sensitivity index (ISI), is connected to the input of the downstream output module for the incretin sensitivity index (12), and wherein the output module for the incretin sensitivity index, after its individual assessment of the incretin sensitivity index, as carried out by itself, produces at its output the data signal for the assessed incretin sensitivity index (ISI_{B}) as a process result of the automated insilico simulation strategy.
